(19) United States
(12) Patent Application Publication
(10) Pub. No.: US 2023/0033235 A1
(43) Pub. Date: Feb. 2, 2023

(54) NPTN65 IS A NEW TARGET FOR THE TREATMENT OF TAUOPATHIES

(71) Applicants: INSERM (INSTITUT NATIONAL DE LAS SANTÉ ET DE LA RECHERCHE MÉDICALE, Paris (FR); CENTRE NATIONAL DE LA RECHERCHE SCIENTIFIQUE (CNRS), Paris (FR); SORBONNE UNIVERSITÉ, Paris (FR)

(72) Inventors: Thierry LEVEILLARD, Paris (FR); Xin MEI, Neuilly-sur-Sein (FR); Mariangela CORSI, Paris (FR)

(21) Appl. No.: 17/791,625

(22) PCT Filed: Jan. 8, 2021
(86) PCT No.: PCT/EP2021/050298

§ 371 Date: Jul. 8, 2022

(30) Foreign Application Priority Data
Jan. 8, 2020 (EP) 20305008.3

(51) Int. Cl. G01N 33/68

(52) U.S. Cl. CPC G01N 33/6896; G01N 2500/04; G01N 2333/70503

(57) ABSTRACT

The NXNL2 gene encodes by alternative splicing for a trophic factor RdCVF2 that enhances the function and the survival of neurons involved in long term memory. Now the inventors demonstrated that the cell surface receptor for the trophic factor RdCVF2 is NPTN65. The set-up of methods that could be used to screen for small molecules, agonists of RdCVF2 signaling in the brain would be suitable for the development of a future metabolic and redox treatment of tauopathies and in particular Alzheimer's disease.

### FIELD OF THE INVENTION

The present invention is in the field of neurology.

### BACKGROUND OF THE INVENTION

Alzheimer's disease accounts for 60% to 70% of cases of dementia. It is a chronic neurodegenerative disease that usually starts slowly and gets worse over time. The most common early symptom is difficulty in remembering recent events (short-term memory loss). As the disease advances, symptoms can include: problems with language, disorientation (including easily getting lost), mood swings, loss of motivation, not managing self-care, and behavioral issues. As a person's condition declines, she or he often withdraws from family and society. Gradually, bodily functions are lost, ultimately leading to death. Although the speed of progression can vary, the average life expectancy following diagnosis is three to nine years.

In the brains of patients suffering from Alzheimer's disease, TAU protein (a microtubule-associated protein that has a role in assembly and stabilization of microtubules) was found to be hyperphosphorylated, leading to aggregation of the protein and to a decrease in TAU binding to microtubules resulting in cell death. Phosphorylated TAU is also toxic to neuronal cells.

Other neurodegenerative diseases associated with the pathological aggregation of tau have been reported, and are collectively designated as “tauopathies”. Progress is being made in understanding the mechanisms underlying tauopathies such as Alzheimer's disease.

Recently the involvement of the Nxnl2 gene in Alzheimer's disease was identified, based the abnormal behavior of the Nxnl2−/− mouse. The aged Nxnl2−/− mice have visual and olfactory deficit (Jaillard et al., Hum Mol Genet. 2012 May 15;21(10):2298-311.), but interestingly, these mice have cognitive deficits that can be scored at 2 months of age before the animals show visual and olfactory dysfunction. These phenotypes were not observed in the Nxnl1−/− mouse in agreement with its expression restricted to the retina. The Nxnl2−/− mouse is hyperactive as demonstrated by the open field test, and has increased anxiety as shown by the elevated plus maze test. This mouse has additional deficits in working memory seen in the Y maze test, contextual memory deficit as seen in fear conditioning, and in spatial memory in the Morris water maze test. However, this mouse has no motor deficit as judged by the rotarod test.

As for RdCVFL, one of the products of the Nxnl1 gene, TAU interacts with the thioredoxin-like protein RdCVF2L, and not with the trophic factor RdCVF2. RdCVF2L inhibits TAU phosphorylation (Elachouri et al., 2015 Free radical biology & medicine 81, 22). By 18 months of age, astrogliosis can be observed in the hippocampus of the Nxnl2−/− brain. At the same age, the analysis of whole brain extracts shows presence of aggregates of TAU as seen by filter binding assay, as well as oligomeric forms of TAU. While the expression of TAU is not modified by the inactivation of the Nxnl2 gene, TAU is phosphorylated in the brain of the Nxnl2−/− mouse as shown using two distinct anti-phosphoTAU antibodies, AT8 and AT100. Interestingly, the expression of NXNL2 is reduced by 48% in the frontal cortex of patients deceased from Alzheimer's disease as compared to age-matched controls.

### SUMMARY OF THE INVENTION

As defined by the claims, the present invention relates to methods for screening a plurality of test substances for use as drugs for the treatment of tauopathies

### DETAILED DESCRIPTION OF THE INVENTION

The NXNL2 gene encodes by alternative splicing for a trophic factor RdCVF2 that enhances the function and the survival of neurons involved in long term memory. Now the inventors demonstrated that the cell surface receptor for the trophic factor RdCVF2 is NPTN65. The set-up of methods that could be used to screen for small molecules, agonists of RdCVF2 signaling in the brain would be suitable for the development of a future metabolic and redox treatment of tauopathies and in particular Alzheimer's disease.

The object of the present invention relates to a method for screening a plurality of test substances for use as drugs for the treatment of a tauopathy comprising the steps consisting of (a) testing each of the test substances for its ability to activate the NPTN65 signaling pathway in a cell and (b) positively selecting the test substances capable of activating said signaling pathway.

As used herein, the term “tauopathy” has its general meaning in the art. It refers to the class of neurodegenerative diseases associated with the pathological aggregation of tau protein in the brain. Tauopathies include, but are not limited to, Alzheimer's disease, traumatic brain injury, frontotemporal dementia, including the subtype of frontotemporal dementia and Parkinsonism linked to chromosome 17 (FTDP-17), progressive supranuclear palsy, corticobasal degeneration, Pick's disease, and agyrophilic grain disease. In a particular embodiment, said tauopathy is selected from the group consisting of Alzheimer's disease and traumatic brain injury.

As used herein, the term “NPTN65” refers to the isoform 2 of neuroplastin. An exemplary amino acid sequence is represented by SEQ ID NO:1.

>sp|Q9Y639|NPTN_HUMAN Neuroplastin OS = Homo
sapiens OX = 9606 GN = NPTN PE = 1 SV = 2
SEQ ID NO: 1
MSGSSLPSALALSLLLVSGSLLPGPGAAQNAGFVKSPMSETKLTGDAFE
LYCDVVGSPTPEIQWWYAEVNRAESFRQLWDGARKRRVTVNTAYGSNGV
SVLRITRLTLEDSGTYECRASNDPKRNDLRQNPSITWIRAQATISVLQK
PRIVTSEEVIIRDSPVLPVTLQCNLTSSSHTLTYSYWTKNGVELSATRK
NASNMEYRINKPRAEDSGEYHCVYHFVSAPKANATIEVKAAPDITGHKR
SENKNEGQDATMYCKSVGYPHPDWIWRKKENGMPMDIVNTSGRFFIINK
ENYTELNIVNLQITEDPGEYECNATNAIGSASVVTVLRVRSHLAPLWPF
LGILAEIIILVVIIVVYEKRKRPDEVPDDDEPAGPMKTNSTNNHKDKNL
RQRNTN

In some embodiments, the screening method of the present invention involves measuring the binding of a test substance to NPTN65, or to cells or membranes bearing NPTN65, or a fusion protein thereof by means of a label directly or indirectly associated with the test substance.

In some embodiments, the method may involve contacting cells expressing NPTN65 with the test substance, and measuring the NPTN65 mediated activity, and comparing the cellular response to a standard cellular response. Typically, the standard cellular response is measured in presence of RdCVF2 wherein a similar or even an increased cellular response over the standard indicates that the test substance is capable of activating the NPTN65 signaling pathway. The determination of the activation of the NPTN65 signaling pathway can be assessed by determining any assay well known in the art. For instance, the neuron viability may be measured. In some embodiments, glucose uptake and/or aerobic glycolysis may be measured wherein an increase of said parameters indicates that the test substance is capable of activating the NPTN65 signaling pathway.

Thus in some embodiments, the screening method of the present invention comprises the steps consisting of: a) providing a plurality of cells expressing NPTN65 on their surface: b) incubating said cells with a test substance; c) determining whether said test substance binds to and activates NPTN65; and d) selecting the test substance that binds to and activates NPTN65.

In some embodiments, such screening methods involve providing appropriate cells which express NPTN65 on their surface. In particular, a nucleic acid encoding NPTN65 may be employed to transfect cells to thereby express the receptor of the present invention. Such a transfection may be accomplished by methods well known in the art. In some embodiments, neurons are preferably used since the naturally expressed the receptor at their surface. In some embodiments, neurons are selected from the group consisting of cone photoreceptor, neurons, retina cells, retinoblastoma and other immortalized neuronal cell lines of any species (mouse, human . . . ).

The test substance of the present invention may be selected from a library of substances previously synthesized, or a library of substances for which the structure is determined in a database, or from a library of substances that have been synthesized de novo. The test substance may be selected from the group of (a) proteins (including antibodies) or peptides, (b) nucleic acids and (c) organic or chemical substances.

The test substances that have been positively selected may be subjected to further selection steps in view of further assaying its properties for the treatment of tauopathies. For example, the test substances that have been positively selected may be subjected to further selection steps in view of further assaying its properties on animal models for tauopathies.

In some embodiments, the substances selected by the above mentioned screening method may be used in the treatment of tauopathies. For example, therapeutic treatments include the reduction or amelioration of the progression, severity and/or duration of tauopathies, or the amelioration of one or more symptoms (specifically, one or more discernible symptoms such as loss of memory) of tauopathies, resulting from the administration of at least one substance selected by the above mentioned screening method. In some embodiments, the therapeutic treatment includes the amelioration of at least one measurable parameter of a tauopathies. In some embodiments, the therapeutic treatment includes the inhibition of the progression of a clinical manifestation of tauopathies (e.g. loss of memory).

In some embodiments, the substances selected by the above mentioned screening method may be used in a prophylactic treatment. The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

Typically, the substances selected by the above mentioned screening method are administered to the subject in an effective amount. As used herein, an “effective amount” refers to an amount sufficient to elicit a therapeutic benefit. The precise amount of compound administered to a subject will depend on the mode of administration, the type and severity of the disease and on the characteristics of the subject, such as general health, age, sex, body weight and tolerance to drugs. The skilled artisan will be able to determine appropriate dosages depending on these and other factors. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between approximately 0.01 to 100 mg/kg body weight/day for therapeutic or prophylactic treatment.

Furthermore, the invention relates to the use of NPTN65 polypeptide in a method for screening a drug for the treatment of tauopathies.

The invention will be further illustrated by the following figures and examples. However, these examples and figures should not be interpreted in any way as limiting the scope of the present invention.

### FIGURES

FIG. 1: RdCVF2 interacts specifically with neuroplastin 65. A. Schematic representation of the single-pass transmembrane proteins NPTN65 and NPTN55 encoded by alternative splicing of the NPTN gene. Notice the presence of a third immunoglobulin domain in NPTN65. B. Schematic representation of the coding exons of the NXNL2 gene and the two proteins product made by alternative splicing. The intron retention and the in-frame stop codon produced an inactive thioredoxin-like protein, RdCVF2 with a truncated thioredoxin fold. C. Binding between the candidate cell-surface receptors, transiently transfected in COS-1 cells, and the candidate ligands expressed as fusion proteins with the alkaline phosphatase (AP).

### Example

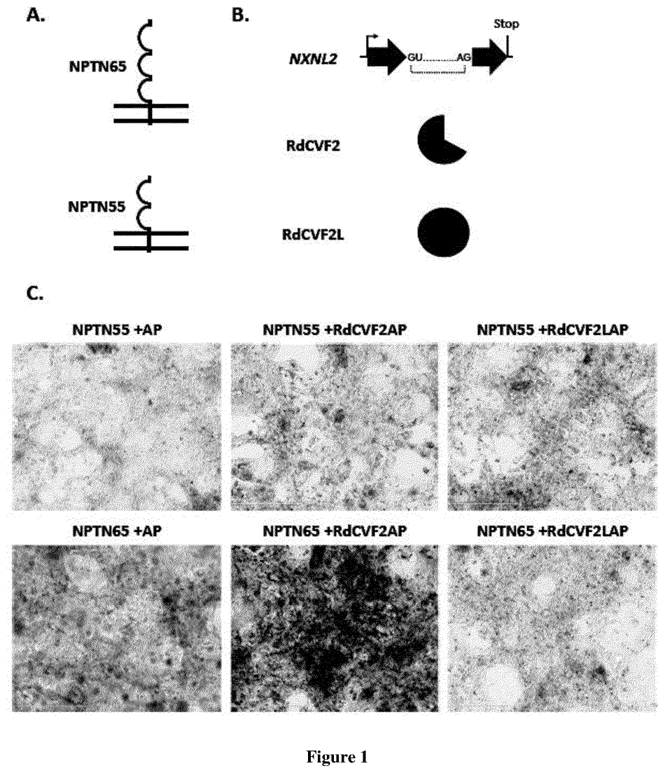

The deficit in long term potentiation of the Nxnl2−/− mouse at 2 months of age was corrected by systemic administration of the combination of RdCVF2 and RdCVF2L using recombinant AAV2/9 carrying a GFP reporter. This result supports the existence of a NXNL2-meditated metabolic and redox signaling in the brain, similar to that of NXNL1 in the retina. In order to validate the hypothesis, we performed a metabolomic analysis of hippocampal slides. We show that the Nxnl2 hippocampus is deficient in glycolysis; glucose-6-phosphate and fructose-16-biphosphate accumulate which is a sign of the reduction of the glycolytic flux. We then demonstrated that the cell surface receptor for the trophic factor RdCVF2 is NPTN65 (FIG. 1). Our results demonstrate that NXNL2 gene encodes by alternative splicing for a trophic factor RdCVF2 that enhances the function and the survival of neurons involved in long term memory through binding to its cell surface receptor, neuroplastin p65 (NPTN65), stimulating glucose uptake and aerobic glycolysis to provide carbohydrate intermediates to build new dendritic spines and/or provide cytoplasmic ATP to regulate the NMDA receptors via the Na/K-ATPase. The long isoform RdCVF2L is a glutaredoxin that interacts with TAU preventing its oxidation, oligomerization, phosphorylation, aggregation and propagation. The function of RdCVF2L is regulated by RdCVF2 and its effect on glucose uptake through the pentose phosphate pathway.

### REFERENCES

Throughout this application, various references describe the state of the art to which this invention pertains. The disclosures of these references are hereby incorporated by reference into the present disclosure.

1. A method for screening a plurality of test substances for use as drugs for the treatment of a tauopathy comprising the steps consisting of (a) testing each of the plurality of test substances for its ability to activate the NPTN65 signaling pathway in a cell and (b) positively selecting the test substances capable of activating said NPTN65 signaling pathway.

2. The method of claim 1 wherein the tauopathy is selected from the group consisting of Alzheimer's disease, traumatic brain injury, frontotemporal dementia, including the subtype of frontotemporal dementia and Parkinsonism linked to chromosome 17 (FTDP-17), progressive supranuclear palsy, corticobas al degeneration, Picks disease, and agyrophilic grain disease.

3. The method of claim 1 wherein the tauopathy is Alzheimer's disease.

4. The method of claim 1, wherein the step of testing comprises measuring the binding of a test substance to NPTN65, or to cells or membranes bearing NPTN65, or a fusion protein thereof by means of a label directly or indirectly associated with the test substance.

5. The method of claim 1 comprising contacting cells expressing NPTN65 with a test substance, measuring an NPTN65 mediated cellular response and comparing the NPTN65 mediated cellular response to a standard cellular response.

6. The method of claim 5 wherein the standard cellular response is measured in the presence of RdCVF2 and wherein a similar or increased NPTN65 mediated cellular response compared to the standard cellular response indicates that the test substance activates the NPTN65 signaling pathway.

7. The method of claim 1 comprising a) providing a plurality of cells expressing NPTN65 on their surface; b) incubating said plurality of cells with a test substance; c) determining whether said test substance binds to and activates NPTN65; and d) selecting the test substance that binds to and activates NPTN65.


SEQUENCE LISTING (1 sequence)

SEQ ID NO: 1 - protein, 398 aa, Homo sapiens
MSGSSLPSALALSLLLVSGSLLPGPGAAQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDDDEPAGPMKTNSTNNHKDKNLRQRNTN